(19) United States
(12) Patent Application Publication
(10) Pub. No.: US 2008/0097184 A1
(43) Pub. Date: Apr. 24, 2008

(54) Magnetically driven dynamic phantom

(76) Inventors: A. Hans Vija, Evanston, IL (US); Johannes Zeintl, Schaumburg, IL (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/524,799

(22) Filed: Sep. 21, 2006

(51) Int. Cl. A61B 5/05; G01R 33/20

(52) U.S. Cl. 600/407; 324/300

(57) ABSTRACT

A phantom according to the invention features a radiotransmissive vessel and a removable closure member, which together define a sealed chamber. A non-magnetic support member extends through a wall of the chamber—preferably through the closure member—and supports a hollow container within the sealed chamber. The support member is pivotally supported through the wall of the chamber by means of a non-leak pivot joint. The support member supports a magnetically drivable element—either a magnet or a ferromagnetic mass—on the opposite end that is not disposed within the chamber. By moving a co-acting magnetically driving element, movement of the support member, and hence the container within the chamber, can be controlled.

### FIELD OF THE INVENTION

The invention relates to phantoms used, for example, to calibrate imaging devices such as PET scanners, SPECT scanners, etc.

### BACKGROUND OF THE INVENTION

In order to prepare images, however, it is necessary to calibrate and ensure the accuracy and proper working order of the nuclear imaging device to be used. This is commonly performed by the use of calibration instruments, known as phantoms. Generally, phantoms are structures having known parameters, which can include, but are not limited to, specific dimensions and/or radiation levels. Accordingly, an image of a phantom produced by a nuclear imaging device can be compared with the actual phantom to determine, for example, image quality, background radiation levels, attenuation information, etc.

In addition to their use as calibration devices, phantoms can also be used for other purposes such as, for example, simulating anatomical conditions for purposes of training individuals to properly use a medical imaging device and/or for training individuals how to read images and/or render diagnoses from the images produced by a medical imaging device. Accordingly, there are various types of phantom and the particular phantom to be used in a simulation depends on a number of factors, which include but are not limited to: specific anatomical area to be studied, e.g., heart, lungs, etc., specific anatomical condition/anomaly to be studied, e.g., normal vs. diseased tissue, etc., and environment, e.g., calibration vs. training. Indeed, phantoms currently range from mere computer software programs, to simple cylindrical devices primarily used for calibration purposes, to more complex mechanical devices that can include pumping mechanisms for mimicking moving body parts, e.g., the human heart.

Phantoms are thus generally known in the art. In general, they include a radiotransmissive body that can be filled with radioactive tracer solution. The phantom may be designed to emulate the uptake or “wash-in” and release or “wash-out” of radioactive tracer by an organ or tissue of interest. Alternatively, the phantom may be designed such that the concentration level of the tracer remains constant. The former type of phantom may be used to simulate the behaviour of diseased tissue, for example. The latter type of phantom, on the other hand, may be used to calibrate the imaging device by imaging a known entity. By moving the phantom within the imaging field and observing how the quality of the image of the phantom varies, non-linearities within the performance of the imaging device can be uncovered and compensated for.

### SUMMARY OF THE INVENTION

The present invention features a magnetically driven dynamic phantom. A phantom according to the invention features a radiotransmissive vessel and a removable closure member, which together define a sealed chamber. A non-magnetic support member extends through a wall of the chamber—preferably through the closure member—and supports a hollow container within the sealed chamber. The support member is pivotally supported through the wall of the chamber by means of a non-leak pivot joint. The support member supports a magnetically drivable element—either a magnet or a ferromagnetic mass—on the opposite end that is not disposed within the chamber. By moving a co-acting magnetically driving element, movement of the support member, and hence the container within the chamber, can be controlled.

### BRIEF DESCRIPTION OF THE DRAWINGS

FIG. 1 is a diagrammatic, side elevation view of a phantom according to an embodiment of the invention.

### DETAILED DESCRIPTION OF EMBODIMENTS OF THE INVENTION

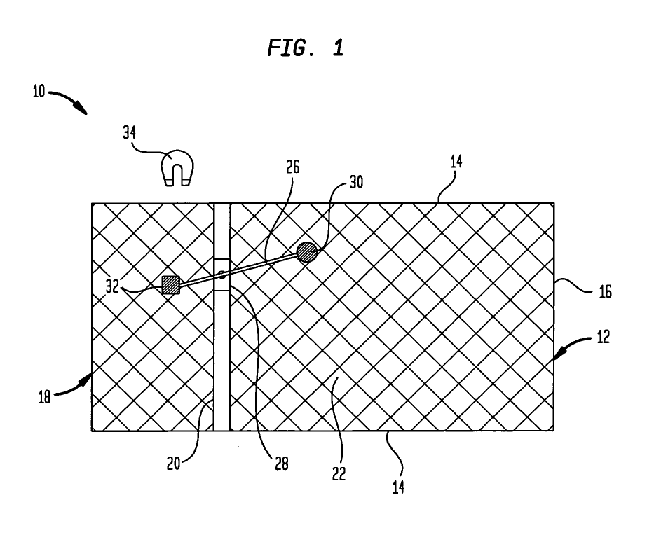

A phantom 10 according to the invention is illustrated in FIG. 1. The phantom includes a vessel 12 that is made from radiotransmissive material such as acrylic. Preferably, the vessel 12 is cylindrical, with cylindrical side wall 14 and circular end wall 16 and open end 18. A closure member 20 is externally threaded (not illustrated) and the open end 18 of the vessel is internally threaded (not illustrated) such that the closure member 20 screws into the open end 18 of the vessel 12. When the closure member 20 is screwed into the open end 18 of the vessel, a sealed chamber 22 is formed within the vessel 12, with the closure member 20 forming one wall of the chamber 22 and the walls of the vessel forming the other walls of the chamber 22.

A support member 26 extends through one of the walls of the chamber 22—preferably through the closure member 20, as illustrated. The support member 26 is pivotally supported in the wall (closure member 20) by means of a leak-proof seal joint 28. The seal joint 28 is configured to allow the support member 26 to pivot up and down, back and forth, left and right, etc., and to swivel such that the support member 26 sweeps a cone.

A hollow container 30, which is formed from radiotransmissive material such as acrylic, is supported on the end of the support member 26 that is disposed within the chamber 22. The hollow container 30 may have any shape that is desired and in use is filled with radioactive solution.

A magnetically drivable element 32 is mounted to the opposite end of the support member 26, i.e., the end that is disposed outside of the chamber 22. The magnetically drivable element 32 may be a magnet, or it may be a mass of ferromagnetic metal that is attracted to a magnet. When a complementarily acting driving element 34 is brought into proximity with the phantom 10 as illustrated, the magnetically drivable element 32 may be drawn toward the driving element 34 (e.g., if the magnetically drivable element 32 is ferromagnetic and the complementarily acting driving element 34 is a magnet or vice-versa), or the magnetically drivable element 32 may be repelled from the complementarily acting driving element (i.e., if the magnetically drivable element and the complementarily acting driving element are both magnets and are arranged with like poles facing toward each other). Hence, with this arrangement, the container 30 can be caused to move within the chamber 22 by moving the complementarily acting driving member around the periphery of the vessel 12 (e.g., by means of a driving motor and armature, not illustrated).

In operation, the container 30 is filled with radioactive tracer material and thus serves as a point source of radiation. Chamber 22 may be filled with a fluid medium such as water. The dynamic, magnetically-driven phantom so constructed may be used to simulate and image functions of the human heart, and/or other anatomical functions and/or characteristics of organs, tissues or bones.

It should be appreciated that the foregoing description of an embodiment of a phantom according to the invention is for illustration purposes only, and that other configurations and embodiments within the spirit of the invention will occur to those having skill in the art. Accordingly, the invention is defined by the following claims.

We claim:

1. A phantom for use in calibrating a radiopharmaceutical emissions-based imaging device, comprising:
a radiotransmissive vessel and a removable closure member, which together define a chamber;
a non-magnetic support member extending through a wall of the chamber, said support member being pivotally and/or swivelingly supported by means of a leak-proof seal joint;
a radiotransmissive container disposed on one end of said support member within the chamber; and
a magnetically drivable member disposed on an opposite end of said support member outside of said chamber.

2. The phantom of claim 1, wherein the wall through which said support member extends is constituted by said closure member.

3. The phantom of claim 1, wherein said radiotransmissive container is filled with a radioactive tracer material.

4. The phantom of claim 1, wherein said radiotransmissive vessel is filled with a fluid medium.

5. The phantom of claim 4, wherein said fluid medium comprises radioactive water.

6. Apparatus using the phantom of claim 1, further comprising a magnetic drive for controlling said magnetically drivable member.
